(19) United States
(12) Patent Application Publication
(10) Pub. No.: US 2023/0077872 A1
(43) Pub. Date: Mar. 16, 2023

(54) Wavelength Supplemented Light Bulbs and Lighting Fixtures

(71) Applicant: University of Cincinnati, Cincinnati, OH (US)

(72) Inventors: Elke Buschbeck, Cincinnati, OH (US); Ilya Vilinsky, Cincinnati, OH (US); Herbert Buschbeck, Wien (AT)

(73) Assignee: University of Cincinnati, Cincinnati, OH (US)

(21) Appl. No.: 17/799,556

(22) PCT Filed: Feb. 15, 2021
(86) PCT No.: PCT/US21/18106

§ 371 Date: Aug. 12, 2022

Related U.S. Application Data
(60) Provisional application No. 62/975,823, filed on Feb. 13, 2020.

(51) Int. Cl. H05B 47/165; H05B 45/10; H05B 45/20; F21S 10/02

(52) U.S. Cl. CPC H05B 47/165; H05B 45/10; H05B 45/20; F21S 10/023; A61N 5/0616

(57) ABSTRACT

The present invention involves a programmable or static multiwavelength LED lighting fixture that is capable of emitting wavelengths of light both for visual perception and other key physiological processes. The lighting fixture produces emitted spectra, wherein one portion of the emitted spectra serves visual perception and at least one portion of the emitted spectra is selected from the group consisting of 290-315 nm; 360-400 nm; 470-490 nm; and 600-1400 nm.

### CROSS REFERENCE TO RELATED APPLICATIONS

The application claims priority to U.S. Provisional Application Ser. No. 62/975,823, filed Feb. 13, 2020, which application is hereby incorporated by reference in its entirety.

### TECHNICAL FIELD

The present invention relates to light bulbs and lighting fixtures that provide specific spectral content to enhance specific physiological processes

### BACKGROUND

The lighting industry has developed a variety of relatively energy-conserving light sources, specifically optimized for the process of vision—seeing our environment through our eyes. However, it also has become clear that many other physiological processes in our body can be influenced by specific wavelength of light, that tend to be absent or insufficient in many of the current light sources. Since our physiology evolved outdoors, under the strong and comprehensive light spectrum of the sun, it is not surprising that treatment with light spectra that are under-represented in normal artificial light environments can yield a variety of positive health effects. Therefore, a strong need exists for the development of spectrally rich, static and dynamic light sources that provide benefits beyond our requirements for visual perception.

### SUMMARY OF THE INVENTION

The present invention involves a programmable or static multiwavelength LED lighting fixture that is capable of emitting wavelengths of light both for visual perception and other key physiological processes. The lighting fixture produces emitted spectra, wherein one portion of the emitted spectra serves visual perception and at least one portion of the emitted spectra is selected from the group consisting of 290-315 nm; 360-400 nm; 470-490 nm; and 600-1400 nm.

In one embodiment, at least one portion of the spectra emitted by the lighting fixture is 290-315 nm. In another embodiment, at least one portion of the spectra emitted by the lighting fixture is 360-400 nm. In another embodiment, at least one portion of the spectra emitted by the lighting fixture is 470-490 nm. In one embodiment, at least one portion of the spectra emitted by the lighting fixture is 600-1400 nm.

In another embodiment, one portion of the emitted spectra from the lighting fixture serves visual perception and at least two distinct portions of the emitted spectra from the lighting fixture are selected from the group consisting of 290-315 nm; 360-400 nm; 470-490 nm; and 600-1400 nm.

In another embodiment, one portion of the emitted spectra serves visual perception and at least three distinct portions of the emitted spectra are selected from the group consisting of 290-315 nm; 360-400 nm; 470-490 nm; and 600-1400 nm. In one embodiment, one portion of the emitted spectra serves visual perception and the following four other distinct portions of the emitted spectra are produced: 290-315 nm; 360-400 nm; 470-490 nm; and 600-1400 nm.

In one embodiment of the present invention, a system is disclosed that involves:
a. a plurality of LEDs configured to produce light that includes at least two different wavelength ranges, wherein at least one wavelength range assists human vision and at least one wavelength range promotes non-visual physiological processes.
b. a processor configured to generate at least one control signal to control power delivered to one or more of the plurality of LEDs, the processor further configured to change the at least one control signal over time so as to produce from the device at least one lighting change; and
c. a user interface adapted to receive a user input to control operation of the processor.

In another embodiment, at least one wavelength range promoting non-visual physiological processes produced by the system is selected from the group consisting of 290-315 nm; 360-400 nm; 470-490 nm; and 600-1400 nm. In another embodiment, at least one wavelength range promoting non-visual physiological processes produced by the system is 290-315 nm. In another embodiment, at least one wavelength range promoting non-visual physiological processes produced by the system is 360-400 nm. In another embodiment, at least one wavelength range promoting non-visual physiological processes produced by the system is 470-490 nm. In another embodiment, at least one wavelength range promoting non-visual physiological processes produced by the system is 600-1400 nm.

In one embodiment, the user interface of the system is selected from the group consisting of a switch, a dial, a keypad and a touchscreen.

In another embodiment, at least one lighting change is configured as a decrease over time of the 470-490 nm wavelength range during evening hours. In one embodiment, multiple lighting changes are configured to simulate natural spectral changes over the course of a day.

In another embodiment, the duration, intensity or both of the emitted spectra selected from the group consisting of 290-315 nm, 360-400 nm, 470-490 nm, and 600-1400 nm, is independently regulated from the portion of the emitted spectra serving visual perception. In one embodiment, the duration and intensity of the emitted spectra selected from the group consisting of 290-315 nm, 360-400 nm, 470-490 nm, and 600-1400 nm, is independently regulated from the portion of the emitted spectra serving visual perception.

### BRIEF DESCRIPTION OF THE DRAWINGS

FIG. 1. is a graph showing a comparison of the relative contribution of different spectra of light.

FIG. 2A is a graph of a typical spectrum of a commercially available white light bulb illustrating a sharp peak around 450 nm and a broader secondary peak around 580 that is produced by a phosphor.

FIG. 2B is a graph of the spectrum of a light bulb according to the present invention that is enriched in violet light, melanopsin stimulating blue light and deep red light.

FIG. 3A is an image of a prototype light bulb according to the present invention.

FIG. 3B shows one embodiment of the light bulb of the present invention. This image shows how the circuit easily fits into a light bulb that can be used in any conventional light bulb housing.

FIG. 3C is an image showing that the light produced by the lamp of FIG. 3B looks white, just as conventional light bulbs do, but the spectrum is specifically enriched with specific physiologically important wavelengths.

FIG. 4A shows an example spectrum containing nine components.

FIG. 4B shows a dynamic light configuration according to one embodiment of the present invention.

FIG. 4C shows a top view of a lamp according to the present invention showing a heat sink and wires that deliver the adequate current to each LED.

FIG. 4D is an image of an LED array according to the present invention mounted onto the heat sink with a thermally conductive (but electrically insulating) glue.

### DETAILED DESCRIPTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

Certain terminology is used in the following description for convenience only and is not limiting. As used herein, “control signal” means a signal that is sent out to control the operation or state of a device. For example, a “control signal” may be generated by a processor or by a user input through an input module, such as a motion (gesture) sensor, touchscreen or button implemented in hardware or software and provided in the device or may be generated through an input tool, such as a motion sensor, touchscreen, or button implemented in hardware or software provided in, e.g., a remote controller, smartphone, tablet PC, wearable device, or accessory that may be used to remotely control the device, but is not limited thereto.

As used herein, “LED” means any solid-state light source, including light emitting diode(s) (LED or LEDs), organic light emitting diode (s) (OLED or OLEDs), and the like.

As used herein, “lighting change” means an alteration in the type of wavelength range (s) and/or the intensity of the light produced by a plurality of LEDs.

As used herein, “processor” means a hardware, firmware, and/or software machine and/or virtual machine comprising a set of machine-readable instructions adaptable to perform a specific task.

As used herein, “user interface” means one or more elements in a device that interface with the user, permitting the user to receive information or allow the user to control device operation. Non-limiting examples of a user interface include a switch, dial, keypad or touchscreen. Those skilled in the art will recognize that the user interface can include other, or different, input and output elements.

Similar reference numerals will be utilized throughout the application to describe similar or the same components of each of the preferred embodiments of the implant described herein and the descriptions will focus on the specific features of the individual embodiments that distinguish the particular embodiment from the others

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The present invention involves novel light sources that provide benefits beyond our requirements for visual perception. Such light sources may include simple light bulbs that are spectrally enriched as well as dynamic lights that are driven by a processor and contribute important spectral content according to the relative prevalence known from the sun spectrum, or according to specific needs.

One factor that makes sunlight particularly powerful when compared to indoor lighting is that its spectrum on Earth sharply rises near 300 nm and continuously extends into the micron range Both ends of this spectrum are well outside the visual detection capabilities of humans and animals alike, even though some animals can see light that is outside our own range. Most of the spectral content is unnecessary for eyes to see a full complement of colors. The human eye only has three “color-sensitive” cones, which act as wavelength detectors with maximal spectral sensitivities at 420 nm (short), 534 nm (medium), and 563 nm (long), as well as rods, which are more light sensitive with a maximal sensitivity at 498 nm. Our perception of any specific color is fabricated by our brain using the relative activation of the cone photoreceptors. This means that, for example, we do not see blue light around 480 nm with a specific receptor, but instead our visual system deduces its presence from relatively mild stimulations of the short- and medium-wavelength detectors. Consequently, we usually fail to notice the relatively low content of this and many other important wavelengths in apparently “white” light sources, such as typical fluorescent tube lights or white LED light bulbs. When it comes to indoor lighting, our current choices are surprisingly limited and there is a strong need for artificial light sources that are supplemented with physiologically important wavelengths. Newly emerging LED technologies provide an especially promising opportunity to produce energy-efficient lights that serve not only our visual needs, but our physiology as a whole.

Taken together, it is clear that our current indoor lighting sources have been designed only to serve the visual perception of our eyes, neglecting other physiological and psychological needs that are outlined below, and can be remediated by supplementing common light sources with specific, physiologically active spectral content.

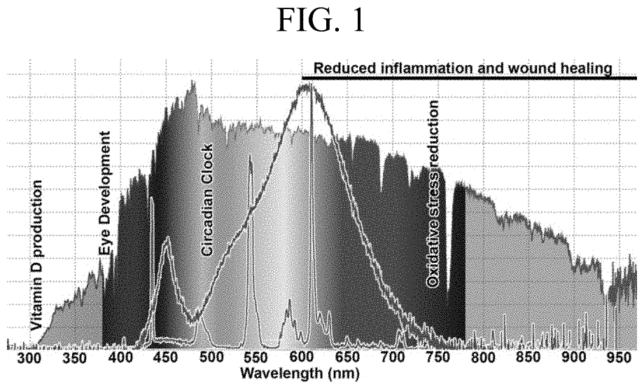

Referring to FIG. 1, the colorful spectrum represents that of sunlight, the green line the spectrum of a typical fluorescent tube light and the blue line that of a soft white LED light bulb. To illustrate the relative spectral content, these graphs have been normalized to each light's maximum. In absolute numbers the sun light would be much higher than the artificial light sources in all wavelengths. Several wavelengths that have been recognized for their physiological importance are labeled in the figure. This figure highlights how critical wavelength content tends to be completely or largely absent from typical light sources.

One embodiment of the present invention involves wavelength supplemented lights, and hence a new type of light sources that are engineered as to specifically include physiologically important wavelengths content that tends to be underrepresented or absent from traditional light sources such as energy saving fluorescent tube lights and LED lighting. Such lights include conventional light sources to which specific wavelengths of light are added in order to facilitate physiological process that do not directly relate to the visual perception of light. Some embodiments of the present invention involve the following four physiologically important types of wavelengths supplements that may be used in healthy lights: 1) UVB (290-315 nm) to enhance Vitamin D production, 2) Violet light (360-400 nm) to reduce Myopia, 3) Melanopsin stimulating blue light (470-490 nm) to properly entrain the circadian clock (daily rhythm), and 4) long wavelength light (600-1400 nm) to reduce inflammation and swelling, help wound healing and mitigate oxidative stress.

Vitamin D production is enhanced through the addition of UVB light (290-315 nm). The importance of a small dosage of UVB light for the production of vitamin D has been widely recognized. At the same time it has become clear that we live in a time and age of an epidemic of Vitamin D deficiency that is particularly severe at higher latitudes and is exacerbated by urban living. There are many negative effects of vitamin D deficiency, including osteoporosis, heart disease, neuropsychiatric disorders, cancer as well as autoimmune and infectious diseases. Perhaps best known to the public is the fact that Vitamin D is necessary to help calcium strengthen bones. This is the reason that Vitamin D supplements typically are administered in combination with calcium supplements. Proper levels of vitamin D are also recognized to be important for proper bone development in children, and it has been noted that low levels of Vitamin D are common among children that present with bone fractures. It also has been established that Vitamin D is an important immunoregulatory factor, including in regard to inflammatory bowel disease, perhaps at least in part through influencing gut bacteria. In addition, data suggests that having sufficient Vitamin D levels likely is protective of several types of cancers. Despite this knowledge, vitamin D deficiency remains a problem even in children (especially girls) in countries with relatively high light levels, especially in the winter and when wearing arm-covering clothing. While exposure to UVB can be dangerous in regard to obtaining sun burns and skin cancer, it also has become clear that vitamin D can be efficiently synthesized by our skin, with only a small fraction of the light that causes sunburn. In fact, depending on the skin type, exposure surface that is available to absorb UV light, the latitude and cloud coverage, the amount of time spent to produce sufficient levels of Vitamin D varies, but generally it only takes a few minutes of sun exposure per day to produce recommended Vitamin D blood levels. The majority of the Vitamin D of most people already comes from casual exposure to sunlight, even though some of the foods (such as Milk or orange juice) are fortified with Vitamin D. This likely is due to very few natural foods containing Vitamin D (except for fish oils). Accordingly, sensible sun exposure has been recommended, and in fact, a study on the elderly has already demonstrated how subliminal ultraviolet B irradiation from an artificial light source can positively affect serum Vitamin D levels. Taken together, there is strong evidence that light sources that supplement relatively low levels of UVB in addition to visual light would be an efficient way to alleviate or eliminate Vitamin D deficiencies, thereby counteracting the silent epidemic of Vitamin D deficiency that has been identified on several continents.

While it seems that most of us would benefit from a little extra UVB at certain times of the year, particularly promising target populations or living conditions include: populations who are living in high latitude—especially if they were native to lower latitude regions; children and adults in which Vitamin D deficiency has been identified; older people who have a more difficult time to produce Vitamin D through their skin; patients with intestinal conditions that interfere with the absorbance of Vitamin D; as a tool to remediate lack of sun exposure during long winter months [a clear seasonal pattern (with a certain lag-time behind light intense seasons) has been established in regard to Vitamin D levels and the US population]; and animal rearing environments.

The dosage of UVB wavelengths largely depends on the intensity of the light source, need, skin type and exposure surface, but ideally the light source would emit a relatively low dosage that is administered over several hours for example in the middle of the day. The light source should never exceed an intensity level that is higher than the wavelength proportion of the sun, if compared to the intensity of the Melanopsin producing blue light band described below. This is important because Melanopsin regulates the pupil diameter, and hence helps preventing overexposure of the retina to this extremely short wavelength content. Appliances also need to contain warnings for consumers not to directly look at the light (just like people never should directly look into the sun). This having said, UVB light intensities of such lamps would be far below the light intensities that are readily encountered by sun light.

There are additional advantages of an artificial UVB source compared to the sun. The dosage can be relatively precisely regulated, and a longer duration—shorter dosage level can be attained. Sun exposure is much more difficult to dose, as cloud coverage, time of the day and latitude make a huge difference in regard to the intensity of UVB radiation that is received. The wavelength can be tuned to maximize Vitamin D production and thus isolated from negative physiological effects that are mediated by slightly different wavelengths.

Light sources prevent Myopia through addition of violet light at 360-400 nm. It has become clear that there has been a relatively recent drastic increase in Myopia or near-sighted ness, especially in Asia. In fact, 96.5% of young adult males are near-sighted in Seoul. This drastic increase makes it clear that in addition to genetic preposition environmental factors play a key role. Most likely these have to do with a shift to indoor living. This notion is supported by intriguing data that shows that adequate exposure to daylight, with its full spectrum, can prevent near-sightedness. More recently an association between exposure to UV light and reduced levels of Myopia have been noted and very recent studies have pointed to the importance of specifically violet light (360-400 nm, the longest wavelength domain of UVA) in that context. One study specifically compared highly myopic patients with intraocular implant lenses that were either transmissive to violet light or largely blocking out violet light. The study clearly showed that the transmission of violet light suppressed the progression of myopia in myopic adults. Another study showed that the development of myopia in children was relatively suppressed when they wore violet transmitting contact lenses compared to children that wore contact lenses that blocked out violet light. A likely candidate for the regulation of eye growth is the Opn5 gene, which encodes a protein called “neuropsin” which is specifically activated by violet light, and is expressed in the eye, brain, testes and spinal cord in humans. While it is likely that not all functions of neuropsin have been characterized to date, there is some evidence that this protein is another important regulator of the circadian and possibly seasonal entrainment. Neuropsin is expressed in certain retinoganglion cells in the eye, and it has been demonstrated to be an important regulator for vascular development in the eye. Intriguingly this regulation is mediated by the dopamine pathway, and in other studies it already has been suggested that dopamine is an important regulator for proper eye growth.

Taken together, scientific literature is emerging that very clearly support the importance of violet light for proper eye development, including the refractive development. At the same time, it is evident that violet light is represented heavily in natural daylight, especially in sunlight, but completely absent from typical indoor lighting. Indoor light sources that supplement violet light therefore would be efficient way to reduce the current prevalence of myopia. Other positive side effects of violet light are anti-inflammatory and immune-modulatory actions. Long-wave UV-A (which basically is violet light) also has been suggested as a synergistic treatment for systemic lupus erythematosus patients, and it is also frequently used to treat a variety of skin conditions such as psoriasis.

Target audiences and environments for myopia prevention wavelengths include: people with a strong familiar history of myopia; people who are already myopic; schools that expose children to artificial light environments during daytime hours (Note here that even if windows are present, they tend to largely block out this critical wavelength domain); any indoor spaces in which especially children spent significant amount of time; and animal rearing environments.

Ideally, the light source would emit a light intensity that is ˜⅓ of the Melanopsin stimulating band described below as to match it to the relative proportions of sun light. This is important because Melanopsin regulates the pupil diameter, and hence helps preventing overexposure of the retina to this relatively short wavelength content.

Melanopsin stimulating blue light (470-490 nm) helps entrainment of the circadian clock. It is widely known that certain wavelengths of blue light are essential for the proper regulation of the circadian clock, yet such light remains largely absent from our indoor lighting. Arguably the most important regulator of our circadian clock is mediated by Melanopsin, which in humans is coded for by the Opn4 gene, which tends to be present in the retinoganglion cells of the eye, but also is found in other parts of the nervous system. Melanopsin is maximally activated by blue light near 479 nm. Its most important role likely is the entrainment of our circadian clocks and associated sleep rhythms. The importance of a proper circadian rhythm has been substantiated by the finding that mutations in important circadian-clock-related genes are linked to mood disorders. Brain activities and dopamine levels also show distinct seasonal variations. It is therefore not surprising that a lack of exposure to sunlight has been recognized as a potential aggravator of depression. Furthermore, blue light therapy has been demonstrated to have positive effects on the sleep cycles and temperature rhythms of people including patients with Alzheimer's disease, which like so many other neurological conditions is accompanied by abnormal sleep and wake patterns. Sleep cycles also become more disrupted in older individuals, a phenomenon that may be of general significance as it has even been established in fruit flies recently. In addition, it is noteworthy to mention that a proper pupillary reflex is depended on stimulating melanopsin, so 470-490 nm supplemented light would allow eyes to regulate the intensity in more natural ways, at least if the intensity of this wavelength band is properly adjusted to the other spectral content.

Taken together it has become clear that we could benefit from proper lighting or even targeted light therapy to keep our circadian clocks better entrained and allow our eyes to better adjust to brightness levels. This need is particularly exacerbated by recent energy efficient light sources, such as LED lamps and light bulbs that specifically lack this important wavelength domain (apparent from FIG. 1).

Target audiences and environments for melanopsin stimulating blue light include: people with seasonally dependent mood disorders; people with insomnia and other sleeping disorders; shift workers who need to entrain their clock to unusual hours; schools that expose children to artificial light environments during daytime hours; any indoor spaces in which especially children spent significant amount of time, such as schools and daycare facilities; any indoor spaces in which the elderly spent significant daytime hours such as retirement homes; any indoor spaces that lack outdoor light, to which people are confined, such as hospitals or rehabilitation centers, submarines and space stations; and animal rearing environments. It would be beneficial to use this light source through the majority of daytime hours, but not within a few hours before going to bed.

Light sources that emit long wavelength light (600-1400) to reduce inflammation and swelling, help wound healing and mitigate oxidative stress. As outlined in this section, a strong body of literature has emerged that outlines specific positive health effects from red, deep-red and near infrared light illumination. These are wavelengths that span from visible red light into infrared, and tend to be represented strongly in daylight, especially in sunlight, but are largely absent in fluorescent bulb and LED-based modern indoor lighting.

Perhaps most exciting here is relatively recent evidence that certain wavelengths of light can influence oxidative stress in beneficial ways. This is important because oxidative stress has emerged as a major contributing factor to many diseases, including cancer and neurological diseases. It is the longer wavelengths of light (red, near-infrared (NIR), or infrared (IR)) that are most often associated with positive health effects, with beneficial mechanisms that include influencing ATP production, nitric oxide signaling, vasodilation, and anti-inflammatory responses that are mediated through changes in transcriptional regulation. While red light therapy has long been used by non-allopathic medical practitioners and has become popular in veterinary medicine, a recent boom in physiological studies has started to draw increased general attention to this practice, and we are starting to understand specific beneficial physiological influences of Low-Level Laser (Light) Therapy. For example, a reduction in reactive oxygen species and improved memory in a mouse model for Alzheimer's disease were observed recently under the application of 630 nm light. Red and NIR light can penetrate deep into tissue, even through the skull, and transcranially administered light has been reported to be potentially beneficial for major depressive disorder. A meta-study on the use of low-level laser therapy with red or NIR light highlighted the many benefits of this treatment for musculoskeletal pain. Here, it was noted that the correct dosage is critical, and strict intensity and timing guidelines have been established.

A particularly exciting recent study reported that, in an animal model, specific wavelengths of NIR light drastically reduce cell mortality in brain cells after an artificially induced heart attack. The unexpected but significant finding of this work was that the neuro-protective effect likely is due to a slowing down of the rate-limiting cytochrome oxidase C enzyme of mitochondria, presumably by allowing it to “catch up” with oxygen production without producing excessive amounts of oxygen radicals. Significantly, certain wavelengths of NIR and IR light differentially influenced this enzyme, indicating that precise spectral content of the illumination source is particularly important. Specifically, while 740 and 950 nm light slowed down the action of cytochrome oxidase C, 850 nm IR light sped it, and hence mitochondrial function, up, in this case leading to enhanced neural cell death. This study clearly demonstrated how the application of specific long wavelengths light supplements can have very specific physiological consequences. Taken together, many studies clearly show health benefits from the application of specific long wavelength light, which tends to be absent from indoor lighting, especially from modern, energy saving light sources.

Target audiences and environments for long wavelength light include: people prone to muscle pain and inflammation; people who are not able to experience sun light for a significant time period; home environment; any indoor spaces in which the elderly spent significant daytime hours such as retirement homes; any indoor spaces that lack outdoor light, to which people are confined, such as hospitals or rehabilitation centers submarines and space stations; and animal rearing environments.

It has been established that there is a biphasic dose response to long wavelength light therapies. If insufficient light is supplied, there is no response, but if too much light is applied then the response may be inhibited. The exact timing hence depends on the specific wavelength content and intensity, but if light supplements are relatively strong then likely a relatively short-term supplement (an hour or less) would be most beneficial.

Taken together, these and other studies highlight the importance of paying attention to the content of specific colors of light as well as the corresponding intensities. As with pharmaceuticals, the devil here seems to be in the dose, and to make it more complicated, which dosages of certain wavelength might be beneficial, also can depend on the content of other wavelengths in the same light source. So, in some cases it likely is important to maintain certain ratios of wavelengths content. One way to construct new lighting is to keep light sources dynamic, with the wavelength content varying at different times of the day to align them with dosage recommendation. Such targeted lighting sources could become important in preventative and management care, as they would support homeostatic (steady-state physiology) mechanisms. For instance, in the future, it might become standard for many elderly, but especially Alzheimer's patients to receive blue light in the late afternoon to help maintain or reestablish healthy sleep rhythms, while receiving targeted doses of red light at other times to help improve memory and reduce inflammation. This example highlights the importance of, and need for, dynamic lighting and appropriate timing of the application of certain light spectra. The solar spectrum changes throughout the day, shifting from shorter to longer wavelengths. Industry and architecture have already recognized these differences and smart lights have become available that can change the color temperature (spectral content as perceived by the eye) according to the time of day or desired ambience. However, currently such lights are designed for visual perception only, and there is a need to extend lighting to other important aspects of human physiology. In addition, improved lighting environments are important for animal rearing.

Static spectrally enhanced LED lights are light sources that include light supplements as outlined above. The simplest form of such lights would be specialty light bulbs, but other LED lamp configurations that combine common use lighting with specific physiological supplements are also possible.

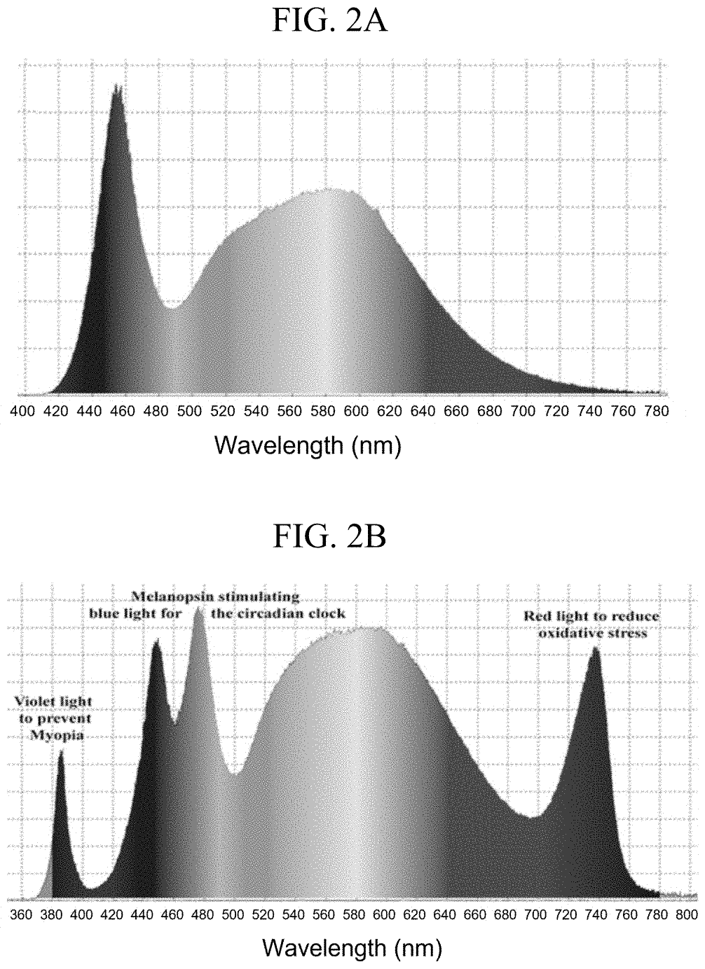

There are many ways to build “healthy light bulbs”, which look just like other light bulbs, but contain physiologically important wavelength content. FIG. 2A shows the spectrum of a commercially available white LED light bulb. It shows how the white-appearing LED spectrum is composed of a primary peak, in this case around 450 nm and a secondary peak that is produced by a phosphor that absorbs some of the light from the primary peak and emits the absorbed energy as longer wavelength light. The emitted light spectrum lacks or is very low in regard to the spectra that are supplemented by specific LEDs in a healthy light bulb. This is illustrated in the spectrum of our prototype FIG. 2B, which has additional peaks around 385 (to help eyes to develop correctly), 480 nm (to help our bodies to set appropriate night/day rhythms and to allow our pupils to better respond to actual light levels) and 730 nm (to help mitigate oxidative stress, and reduce inflammation among other functions).

The healthy light bulbs of the present invention may be fabricated in many ways. The specific designs described herein are for illustrative purposes only and are not intended to be limiting. An important aspect of the light bulbs of the present invention is that a general use light is supplemented with a wavelength spectrum that specifically targets at least one physiological property through the supplementation of a wavelength spectrum.

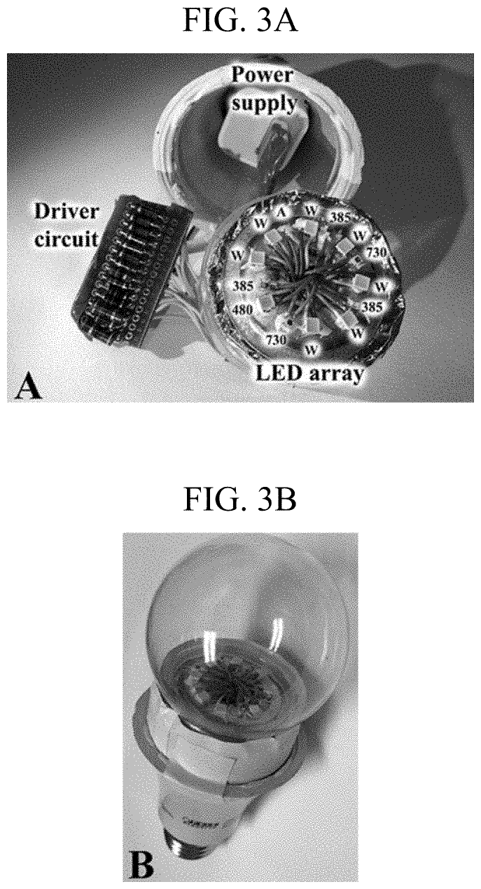

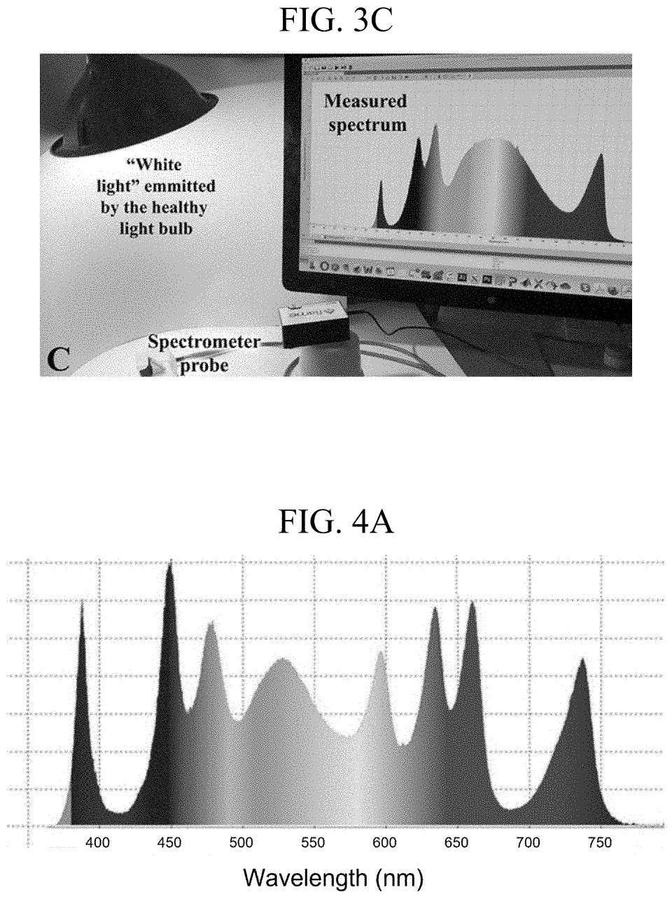

In one embodiment, a lamp of the present invention is shown in FIG. 3A. It has a power supply, a driver circuit and an LED array. In this case, the array is housing 7 white LEDs (W), three myopia reducing 385 nm LEDs, one melanopsin stimulating blue LED (480 nm), two deep red LEDs (730 nm) that are designed to mitigate oxidative stress and reduce inflammation, and one broader-spectrum amber LED (A) that is contained within the broad peak, and helps to restore a white color to the light source, that otherwise would have a blue tint. A driver circuit that regulates necessary currents for each LED controls the LEDs. While not part of this specific prototype, this driver circuit also could include switches that allow for specific light supplements to be independently turned on or off. The circuit board of this prototype is powered by a 5V power supply, and here all LEDs are configured in parallel. Other options are to use higher voltage power supplies and to configure certain LEDs in series.

The circuit easily fits into a light bulb (FIG. 3B) that can be used in a conventional light bulb housing. As FIG. 3C illustrates, the light that is produced by this lamp looks white, just as conventional light bulbs do, but the spectrum is enriched with specific physiologically important wavelengths. This highlights how the lighting system if the present invention can be configured to be visually pleasing in addition to containing physiologically effective wavelength supplements.

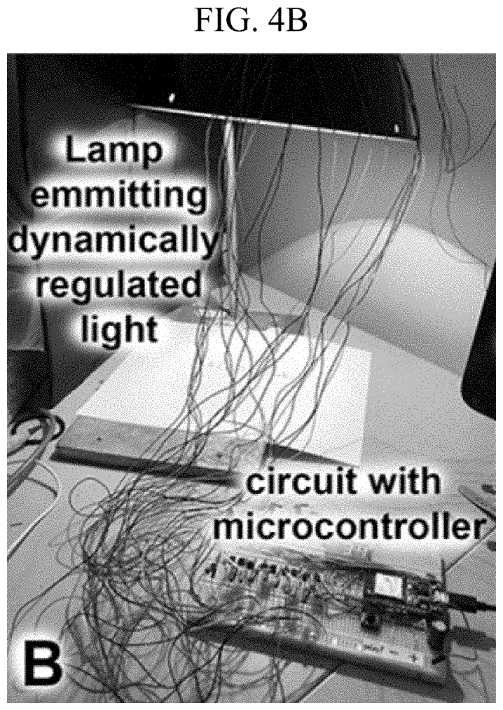

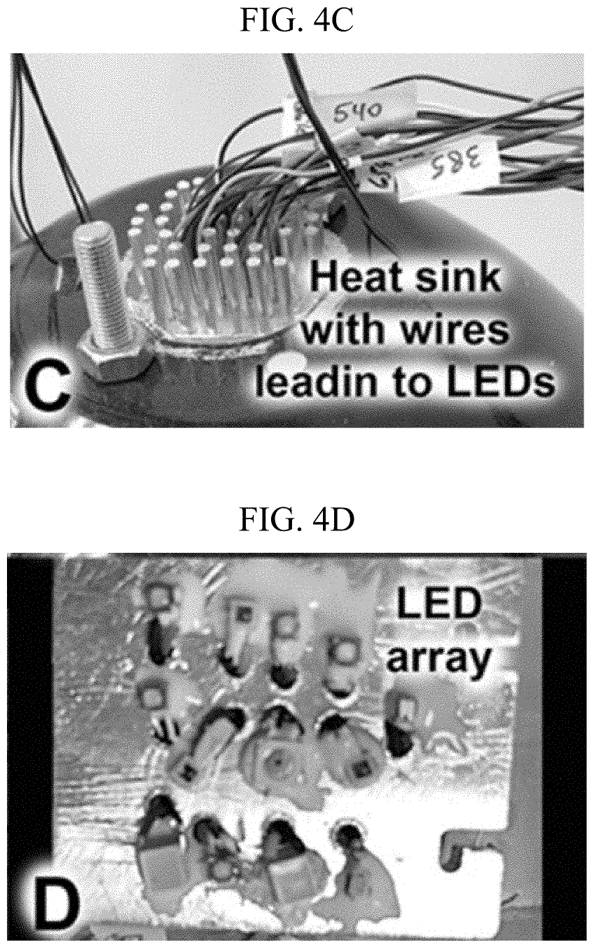

In other light appliances a variety of LED emitters can be dynamically tuned as to achieve specific light spectra according to the momentary need or according to the time of the day. In contrast to the static spectrally enhanced LED lights, these lights are dynamically adjustable through a microprocessor that allows to dynamically adjust the relative intensity of specific wavelengths content. For example, this includes lights that can be programmed so that blue components (including the melanopsin exciting light around 480 nm) can be tuned down during the evening hours. It also includes lights that can be programmed to closely represent outdoor lighting as encountered either during a sunny day or a cloudy day. Such lights also could be programmed as to closely follow the natural daytime dependent spectral change that are known to occur outdoors. Such lighting has many possibilities up to a nearly complete mimic of the outdoor lighting spectrum. For the latter it could be programmed to have a reduction or absence of certain harmful UV components. FIG. 4 illustrates one prototype of such a light source that can be dynamically regulated through a microcontroller. This prototype lamp can produce spectrally rich LED light in a dynamic way. FIG. 4A shows an example spectrum containing 9 components. More extensive microcontrollers can produce a much larger number of peaks, that seamlessly can produce any desired spectrum. FIG. 4B. shows a dynamic light configuration according to one embodiment of the present invention. It has a circuit that contains the microcontroller as well as the lamp that emits a “white” spectrum that is composed of single color LEDs (but also could include “white” LEDs). The microcontroller regulates the light intensity of each LED through pulse-width modulation. FIG. 4C is a top view of the lamp showing a heat sink and wires that deliver the adequate current to each LED. This lamp was used to produce spectra illustrated in FIG. 4A. A lamp was photographed while emitting light that looks white to the eye, but that is composed of multiple single color LED. The micro controller regulated the intensity of individual colored LEDs through pulse-with modulation. FIG. 4D. shows an LED array according to the present invention mounted onto the heat sink with a thermally conductive (but electrically insulating) glue.

In one embodiment of the present invention, the system is configured to produce at least one lighting change as a decrease over time of the 470-490 nm wavelength range during evening hours. In another embodiment, the system is configured to produce at least one lighting change as a decrease or increase over time of a wavelength range that produces a physiological effect.

In another embodiment, multiple lighting changes are configured to simulate natural spectral changes over the course of a day. The spectral changes may follow a “standard day” with a lighting pattern that repeats every day. Alternatively, the spectral changes may be linked to an exterior detection device, such as a light sensor, that produce changes based on actual light conditions in an outdoor area. The outdoor area may be near the lighted space. Alternatively, information may come from an outdoor area in another part of the world.

In another embodiment, the duration, intensity or both of the emitted spectra selected from the group consisting of 290-315 nm, 360-400 nm, 470-490 nm, and 600-1400 nm, is independently regulated from the portion of the emitted spectra serving visual perception. In one embodiment, the duration and intensity of the emitted spectra selected from the group consisting of 290-315 nm, 360-400 nm, 470-490 nm, and 600-1400 nm, is independently regulated from the portion of the emitted spectra serving visual perception.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A programmable or static multiwavelength LED lighting fixture that is capable of emitting wavelengths of light both for visual perception and other key physiological processes, the lighting fixture producing emitted spectra, wherein one portion of the emitted spectra serves visual perception and at least one portion of the emitted spectra is selected from the group consisting of:
a. 290-315 nm;
b. 360-400 nm;
c. 470-490 nm; and
d. 600-1400 nm.

2. The lighting fixture of claim 1 wherein at least one portion of the emitted spectra is 290-315 nm.

3. The lighting fixture of claim 1 wherein at least one portion of the emitted spectra is 360-400 nm.

4. The lighting fixture of claim 1 wherein at least one portion of the emitted spectra is 470-490 nm.

5. The lighting fixture of claim 1 wherein at least one portion of the emitted spectra is 600-1400 nm.

6. The lighting fixture of claim 1 wherein one portion of the emitted spectra serves visual perception and at least two distinct portions of the emitted spectra are selected from the group consisting of:
a. 290-315 nm;
b. 360-400 nm;
c. 470-490 nm; and
d. 600-1400 nm.

7. The lighting fixture of claim 1 wherein one portion of the emitted spectra serves visual perception and at least three distinct portions of the emitted spectra are selected from the group consisting of:
a. 290-315 nm;
b. 360-400 nm;
c. 470-490 nm; and
d. 600-1400 nm.

8. The lighting fixture of claim 1 wherein one portion of the emitted spectra serves visual perception and four other distinct portions of the emitted spectra are produced:
a. 290-315 nm;
b. 360-400 nm;
c. 470-490 nm; and
d. 600-1400 nm.

9. The lighting fixture of claim 1 wherein the duration, intensity or both of the emitted spectra selected from the group consisting of 290-315 nm, 360-400 nm, 470-490 nm, and 600-1400 nm, is independently regulated from the portion of the emitted spectra serving visual perception.

10. A system comprising:
a. a plurality of LEDs configured to produce light that includes at least two different wavelength ranges, wherein at least one wavelength range assists human vision and at least one wavelength range promotes non-visual physiological processes.
b. a processor configured to generate at least one control signal to control power delivered to one or more of the plurality of LEDs, the processor further configured to change the at least one control signal over time so as to produce from the device at least one lighting change; and
c. a user interface adapted to receive a user input to control operation of the processor.

11. The system of claim 10 wherein the at least one wavelength range promoting non-visual physiological processes is selected from the group consisting of:
a. 290-315 nm;
b. 360-400 nm;
c. 470-490 nm; and
d. 600-1400 nm.

12. The system of claim 10 wherein the at least one wavelength range promoting non-visual physiological processes is 290-315 nm.

13. The system of claim 10 wherein the at least one wavelength range promoting non-visual physiological processes is 360-400 nm.

14. The system of claim 10 wherein the at least one wavelength range promoting non-visual physiological processes is 470-490 nm.

15. The system of claim 10 wherein the at least one wavelength range promoting non-visual physiological processes is 600-1400 nm.

16. The system of claim 10 wherein the user interface is selected from the group consisting of a switch, a dial, a keypad and a touchscreen.

17. The system of claim 10 wherein at least one lighting change is configured as a decrease over time of the 470-490 nm wavelength range during evening hours.

18. The system of claim 10 wherein multiple lighting changes are configured to simulate natural spectral changes over the course of a day.

19. The system of claim 10 wherein the duration, intensity or both of the emitted spectra selected from the group consisting of 290-315 nm, 360-400 nm, 470-490 nm, and 600-1400 nm, is independently regulated from the portion of the emitted spectra serving visual perception.

20. The system of claim 19 wherein the duration and intensity of the emitted spectra selected from the group consisting of 290-315 nm, 360-400 nm, 470-490 nm, and 600-1400 nm, is independently regulated from the portion of the emitted spectra serving visual perception.
